(19) United States
(12) Patent Application Publication
(10) Pub. No.: US 2025/0275866 A1
(43) Pub. Date: Sep. 4, 2025

(54) METHOD AND APPARATUS FOR REDUCING VULVOVAGINAL PAIN

(71) Applicant: Pelva Health Corporation, Saint Paul, MN (US)

(72) Inventors: Kelley Anne Satoski, Edina, MN (US); Douglas James Satoski, Edina, MN (US)

(73) Assignee: Pelva Health Corporation, Saint Paul, MN (US)

(21) Appl. No.: 19/066,842

(22) Filed: Feb. 28, 2025

Related U.S. Application Data
(60) Provisional application No. 63/559,299, filed on Feb. 29, 2024.

(51) Int. Cl. A61F 6/08

(52) U.S. Cl. CPC A61F 6/08

(57) ABSTRACT

A vulvovaginal sheath for use in the reduction of vulvovaginal pain and/or discomfort during sexual intercourse and/or penetration. The vulvovaginal sheath comprising at least one hydrogel layer and having a force dissipation and dispersion characteristic enabling a person with provoked or pre-existing vulvovaginal pain or discomfort to engage in sexual intercourse or other penetration while reducing such vulvovaginal pain or discomfort.

### CROSS-REFERENCE TO RELATED APPLICATIONS

This application is being filed on Feb. 28, 2025, as a nonprovisional application and claims the benefit of and priority to U.S. Provisional Patent Application No. 63/559,299, filed Feb. 29, 2024, the entire disclosure of which is incorporated by reference herein in its entirety.

### BACKGROUND

It is estimated by the American College of Obstetricians and Gynecologists that 75% of women will experience pain in the vagina, vulva, and/or vestibule during intercourse at some point in their lives and that at least 28% of women will experience pain chronically (pain that lasts over 6 months) in the vagina and/or vulva during penetration whether that be in the form of sexual intercourse, pelvic exams or tampon insertion (see Chisari C, Monajemi M B, Scott W, Moss-Morris R, McCracken L M. Psychosocial factors associated with pain and sexual function in women with Vulvodynia: A systematic review. Eur J Pain. 2021; 25(1): 39-50. doi: 10.1002/ejp.1668, and Mitchell K R, Geary R, Graham C A, et al. Painful sex (dyspareunia) in women: prevalence and associated factors in a British population probability survey. BJOG. 2017; 124(11): 1689-1697. doi: 10.1111/1471-0528.14518).

There are several conditions that are defined by chronic vulvovaginal pain. These conditions include vaginismus or genito pelvic pain penetration disorder (GPPPD), an involuntary tightening of vaginal muscles impeding one's ability to engage in any form of vaginal penetration; dyspareunia, pain with sexual intercourse caused by but not limited to scar tissue, lack of lubrication, vaginal atrophy, tight pelvic floor muscles, and/or inflammation; vulvodynia, pain or burning in the vagina and/or vulva; and provoked vestibulodynia, chronic pain and discomfort that occurs in the area around the opening of the vagina, inside the inner lips of the vulva commonly experienced in, but not exclusive to, reproductive aged women. The current standard of care and treatment for all of these vulvovaginal pain conditions is to prescribe and/or treat with dilation therapy, an 84-year-old method of increasing vaginal elasticity, control, and capacity. This method is often paired with some form of lubrication for ease of insertion during dilation therapy.

Dilation therapy has been studied to have severely low compliance rates, as low as 25%, and requires the patient to complete therapeutic exercises daily for results (see Lee Y. Patients' perception and adherence to vaginal dilator therapy: a systematic review and synthesis employing symbolic interactionism. Patient Prefer Adherence. 2018; 12:551-560. Published 2018 Apr. 12. doi: 10.2147/PPA.S163273). Typical time to relief with optimal compliance is at least 6 months, but because of low compliance, time to relief averages over 2 years, placing undue strain on patients' mental health and intimate relationships. Additionally, dilators are most often made of inflexible & rigid material that fails to adapt to female anatomy causing undue force on hypertonic pelvic floor muscles. Pain and burning are often experienced in dilation therapy which can reinforce vulvovaginal pain, provoking the psychosomatic “pain-fear cycle”. Dilation therapy fails to provide positive exposure therapy directly to sexual activity, often causing a lack of translation to sexual intercourse with one's partner. For example, a woman may undergo dilator therapy with success but remain unable to have sexual intercourse without severe pain.

The objective of dilation therapy is to “train” the vaginal canal to dilate without vulvovaginal pain and/or to reduce the amount of vulvovaginal pain experienced during such dilation. From this objective, it follows that dilation therapy is designed with the expectation that the person suffering from vulvovaginal pain either cannot or should not undergo sexual intercourse and/or other types of vaginal penetration until they have successfully completed the dilation therapy. As such, existing dilation therapy products devices and methods are not designed to allow vaginal penetration during use or insertion and would therefore prevent the person suffering from vulvovaginal pain from engaging in sexual intercourse or vaginal penetration when a dilator is inserted for use.

As an alternative approach to dilation therapy, traditional lubricants are used to help with insertion and reduce friction during penetration, they often have several drawbacks including drying quickly, requiring reapplication, creating mess for both partners, often leaking out of the vagina, and not guaranteeing full coverage lubrication along the entirety of the vaginal canal and perineum. For people with chronic and/or more severe vulvovaginal pain, typical personal lubricants are often not enough to prevent pain because lubrication does not sufficiently distribute radial forces from the penis and or pelvic muscles. Similarly to liquid lubricants, vaginal suppositories may be inserted prior to intercourse and dissolve into liquid lubricant. This poses several drawbacks including having to insert the suppositories early enough so they have time to dissolve, and often suppositories that degrade in the vaginal canal release large volumes of liquid which can be uncomfortable or flow out of the vaginal canal at inopportune times.

Internal female condoms and male condoms, by contrast, are designed with the objective of preventing pregnancy and/or transmission of communicable disease. Condoms, for example, are traditionally made of synthetic materials that often require additional lubrication. Condoms are also made to be as thin as possible which then fails to provide a proper cushioning and/or pressure dispersion for a person suffering from vulvovaginal pain. Male condoms constrict the penis and move with penetration which continues to aggravate nociceptors in the vulvovaginal region. Internal condoms work similarly but often receive complaints about not staying in place within the vaginal canal. They are also frequently dismissed due to causing discomfort during intercourse, such as unwanted folding, chafing, and a “bag-like” sensation for the male partner.

Both types of condoms do not provide enough padding to reduce pain around the vulva and perineum and generally rely on the use of a finger for insertion which can be difficult for women with vulvovaginal pain conditions due to size, reach, and discomfort with touching their own genitalia.

### SUMMARY

It is therefore an objective of the present invention to provide a method and apparatus to overcome these and additional shortcomings of the prior art for the treatment and/or prevention and/or alleviation of vulvovaginal pain and other related conditions and objectives.

It is another objective of the present invention to provide a method and apparatus to address vulvovaginal pain to enable vaginal intercourse and/or penetration without the prior requirement of a dilation therapy or to allow one to use the present invention in tandem with dilation therapy.

It is an additional objective of the present invention to provide a method and apparatus that addresses the shortcomings of personal lubricants in reducing and/or minimizing vulvovaginal pain during intercourse and/or penetration and/or insertion (e.g.: penetration by sex toy, dilator, tampon, menstrual cup or disk, or medical devices and instruments).

It is yet another objective of the present invention to provide a method and apparatus that addresses the shortcomings of condoms in reducing and/or minimizing vulvovaginal pain during intercourse and/or penetration.

It is yet another objective of the present invention to provide a method and apparatus that reduces vulvovaginal pain and maintains pleasure for both partners by mimicking the natural vaginal mucosa as closely as possible.

It is yet another objective of the present invention to provide a method and apparatus that addresses the shortcomings of condoms in maintaining pleasure for both partners during sexual intercourse through a design that closely mimics the texture, quality, and sensation of healthy vaginal tissue and mucosa, ribbing, and through a non-constrictive design that forces the invention to stay in place during sexual intercourse.

It is yet another objective of the present invention to provide a method and apparatus that addresses the shortcomings of condoms in being made of sustainable and fully biodegradable material through a non-polyurethane, non-plastic, non-latex, and non-rubber, material.

These and additional objectives of the methods and apparatus of the present invention are provided in the detailed description of the present invention.

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon which the embodiments disclosed herein are based.

### BRIEF DESCRIPTION OF THE DRAWINGS

The accompanying drawings, which are incorporated in and constitute a part of the description, illustrate several aspects of the present disclosure. A brief description of the drawings is as follows:

FIG. 1 is a cutaway side view of a vaginal sheath in accordance with one or more embodiments of the present invention.

FIG. 2 is a perspective view of a vaginal sheath in accordance with one or more embodiments of the present invention.

FIG. 3 is a perspective view of a vaginal sheath in combination with an insertion applicator in accordance with one or more embodiments of the present invention.

FIG. 4 is a cutaway side view of an insertion applicator in combination with a sheath in accordance with one or more embodiments of the present invention.

FIG. 5 is a side view of an insertion applicator in accordance with one or more embodiments of the present invention.

### DETAILED DESCRIPTION

Reference will now be made in detail to exemplary aspects of the present disclosure that are illustrated in the accompanying drawings. Wherever possible, the same reference numbers will be used throughout the drawings to refer to the same or like parts.

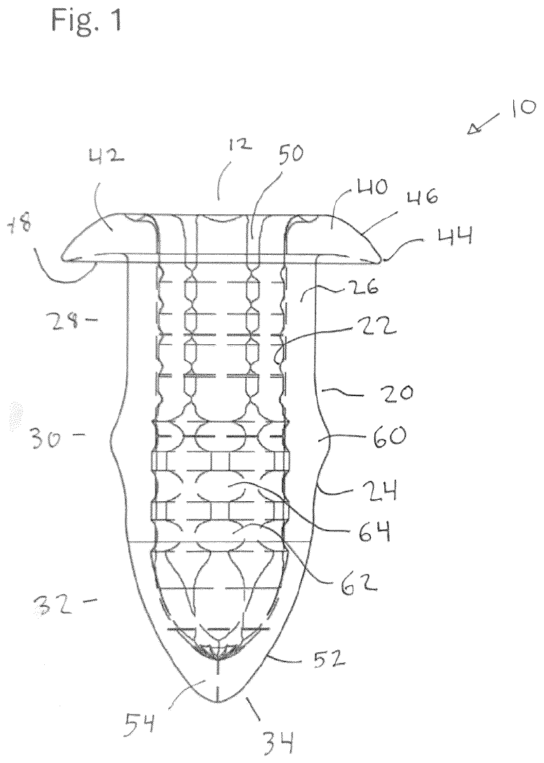

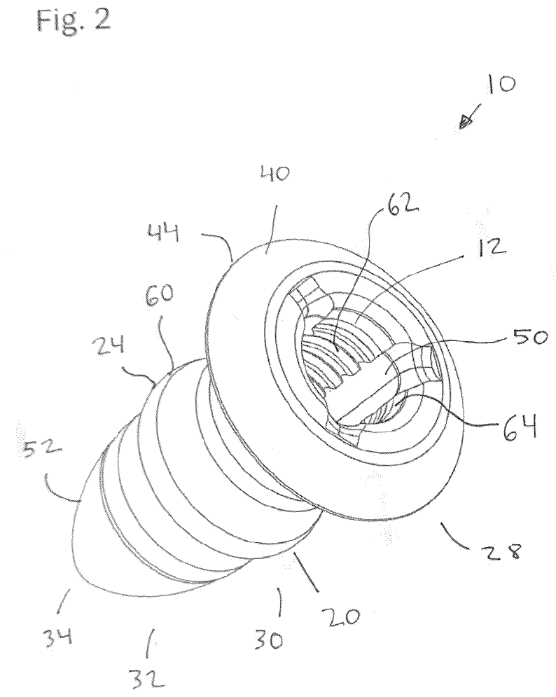

FIGS. 1 and 2 illustrate a vulvovaginal sheath 10 in accordance with one or more embodiments of the present invention. The vulvovaginal sheath 10 described with reference to this and additional embodiments of the present invention is designed for persons who suffer from provoked or pre-existing vulvovaginal pain or discomfort during sexual intercourse and/or vaginal penetration; however, it is understood that anyone may choose to use the vulvovaginal sheath 10 for preventative pain measures or, in accordance with some embodiments of the present invention to simply enhance the vaginal penetration experience or, in accordance with some embodiments of the present invention utilize the present invention as a contraceptive.

The vulvovaginal sheath 10 comprises a generally tubular structure 20 defining an inner wall surface 22 and an outer wall surface 24 and thereby having and defining an inner diameter and an outer diameter, respectively. The tubular structure has a wall thickness 26 defined by the distance between the inner wall surface 22 and the outer wall surface 24. The tubular structure 20 further has a proximal portion 28, a central portion 30 and a distal portion 32.

In the present embodiment, the inner wall surface 22 of the proximal portion 28 defines a plenum. The plenum extends through the central portion 30 of tubular structure 20 to the distal portion 32 of the tubular structure 20. The distal portion 32 of the tubular structure 20 has a closed end portion 34.

As further shown in the present embodiment, a flange 40 extends from at least a portion of the periphery of the proximal portion 28 of the tubular structure 20. The flange 40 (the external portion of the device) has a primary function of keeping the device from being pushed too far into the vaginal canal during use and getting stuck. To accomplish this, the flange 40 has a flange wall thickness 42 is slightly thicker than the wall thickness 26 of the tubular structure 20—the flange wall thickness 42 provides some rigidity and structure. The peripheral shape of the flange 40 of the present embodiment, from an axial perspective with respect to the tubular structure 20, is ovular or circular. The flange 40 also has a larger periphery 44 than the rest of the device 10 to prevent the tubular structure 20 being pulled into the vaginal canal and to maintain a predetermined positioning of the distal portion 28 and central portion 30 of the sheath 10 within vaginal canal. The flange 40, in the present embodiment has a wall thickness 42 that includes a taper 46 as it extends from the opening 12 of the sheath 10 to the periphery 44 of the flange 40, so as to reduce the volume of the flange 40 and create a more subtle appearance. The flange 40 has an angled or concave under surface 48 so as to prevent flopping outwards of the flange 40 during placement and use. The flange 40 is dimensioned to have a peripheral flange 44 diameter and a flange wall thickness 42 each of which, either alone or in combination, serve to disperse and dissipate force along the outer vulvar and vestibular sections of the pelvic region during placement, penetration or intercourse to prevent and reduce pain in the anatomical regions capable of being covered by the flange 40.

The vulvovaginal sheath is made of a material that is deformable but capable of maintaining the general integrity of the various embodiments of the present invention, wherein examples of such materials and alternative materials and shapes are discussed in more detail below.

The vulvovaginal sheath 10 is intended for use during sexual intercourse and/or other types of vaginal penetration and/or insertion. As such, the vulvovaginal sheath 10 is dimensioned such that the vulvovaginal sheath can be inserted in a vaginal canal prior to sexual intercourse and/or penetration and/or insertion. By way of example, but not intended to be limiting as to scope, the outer diameter of the vulvovaginal sheath 10 is dimensioned to be deformably insertable into the vaginal canal in that it collapses in on the plenum to reduce the outer diameter, therefore making insertion easier. The undeformed outer diameter of the tubular structure 20 may be in the range of 10 mm to 50 mm depending on the deformability of the particular material being used for the vulvovaginal sheath 10, as will be discussed in more detail below. The deformability allows the sheath 10 to expand in both length and diameter to accommodate a variety of penetrative objects and object sizes (penis sizes, dilator sizes, medical instrument sizes etc.). The undeformed inner diameter of the tubular structure 20 is dimensioned to be deformable and capable of enabling sexual intercourse and/or penetration, specifically, allowing the penis and/or penetrating element to be received within the plenum of the tubular member. As such, the inner diameter of the tubular member may be in the range of 10 mm to 50 mm depending on the deformability of the material being used for the construction of the vulvovaginal sheath and may be deformably stretchable when an object is inserted therein.

The wall thickness 26 of the tubular structure 20 of the vulvovaginal sheath 10 is dimensioned to dissipate forces that are created during the act of sexual intercourse and/or penetration and is also dependent on the durometer of the material chosen. Initiating forces, such as frictional forces created by the penis during sexual intercourse, are translated to the inner wall surface 22 of the tubular structure 20 and are dissipated and dispersed by the material properties of the tubular structure 20 thereby reducing the forces and/or concentration of forces experienced on the vaginal wall. Conversely, forces initiating from the vaginal wall are dissipated and dispersed by the material properties of the tubular structure 20 prior to being translated to the interior wall surface 22. The material properties of the sheath 10 for enabling the dissipation and dispersement of forces include one or more of the wall thickness 26 of the sheath 10, the durometer of the sheath 10, the lubriciousness of the sheath 10, the material composition of the sheath 10 and additional material properties as will be discussed in more detail below.

A person having a condition of vulvovaginal pain may have one or more spots of scar tissue, inflammation, or sensitivity on a localized position of the vaginal wall, vestibule, and/or vulva. The vulvovaginal sheath, by virtue of the wall thickness 26 and/or other material properties, provides the force dissipation to reduce the translated force that the anatomical source of pain that a person having this condition is subjected to, as compared to the forces that would otherwise be delivered to the anatomical source without the use of the vulvovaginal sheath 10. Likewise, the forces that the anatomical source may otherwise exert on the penis during intercourse without the use of the vulvovaginal sheath 10 are also dissipated and dispersed by the vulvovaginal sheath 10 prior to being translated to a sensation on the penis. It should be understood that dissipation and dispersion have slightly different meanings. In this case the sheath 10 distributes forces over a larger area (pressure=force/area), thus lowering pressure on small sections of tissue, including the tissue of the anatomical source of pain. But certainly, the sheath 10 also dissipates forces by absorbing some of the kinetic energy during the act of squeezing, such as by the vaginal muscles or the act of penetration and/or sexual activity. Both force dissipation and force dispersion help to relieve sharp increases in pressure on tissue that ultimately might otherwise produce a sensation of pain. As such, the wall thickness 26, 42 in tandem with the qualities of the material chosen of the sheath 10 is dimensioned such that initiating forces on the inner wall 22 are dissipated when translated to the outer wall 24, and conversely initiating forces on the outer wall 24 are dissipated when translated to the inner wall 22, and thereby reducing the magnitude of such forces on the vaginal wall, or conversely the penis, from a first initiating force to a reduced dissipated receiving force, and vice versa. By way of example, the wall thickness 26 of the tubular structure 20 may be at least 1 mm to 15 mm depending on the material used in constructing the sheath 10 and/or the amount of force dissipation that a given person suffering from vulvovaginal pain or discomfort requires. Likewise, the thickness 42 of the flange 40 is dimensioned to provide a predetermined amount of force dissipation on the anatomy exterior to the vaginal canal.

While the above discussion focused on the material property of wall thickness 26, 42 as providing the force dissipation and dispersion function, discussion now turns to material design and selection, either alone or in combination with material wall thickness 26, 42, to achieve the force dissipation requirements to alleviate vulvovaginal pain or discomfort.

In one embodiment of the present invention, the vulvovaginal sheath 10, as described above with reference to FIGS. 1 and 2, is made of a hydrogel material. The hydrogel material in accordance with the present invention is a soft, deformable, elastic hydrogel that can be formed in accordance with the general shape, functions and dimensions as described above.

The hydrogel material may include any number of ingredients, including but not limited to the following categories of ingredients either alone or in various combinations of one or more such categories of ingredients: aqueous solvent, polysaccharide, preservative, co-solvent, cross-linking agents and stabilizers, pH modifying agents, active ingredients. Example embodiments incorporating some or all of these categories of ingredients are included below but are intended to be illustrative and not intended to be limiting in accordance with the scope and spirit of the present invention:

### Hydrogel Composition Ingredients

| Ingredient | Representative Materials | Amount (% w/w) |
|---|---|---|
| Aqueous Solvent | Water | At least 80% |
| Polysaccharide | Collagen, cellulose | .1-10% |
| | derivatives, K carrageenan, | |
| | Konjac Gum, agar, agarose, | |
| | alginate, gelatin, other | |
| | forms of Carrageenan, | |
| | hyaluronic acid, chitin, | |
| | chitosan, pectin, Xanthan | |
| | Gum, Locust Bean Gum, | |
| | Gellan Gum, Guar Gum, | |
| | Gum Arabic | |
| Preservative | Potassium sorbate, Sodium | 0.05-2% |
| | benzoate, | |
| | Ethylhexylglycerin | |
| | combined with | |
| | phenoxyethanol. | |
| Co-Solvent | Glycerol, PEGs | 1-10% |
| Cross- | NaCl, Calcium lactate, | 0.1-5% |
| Linking/ | calcium chloride, potassium | |
| Strengthening | chloride etc. | |
| Agent | | |
| Other | pH modifying agents, active | 0.01-10% |
| | ingredients (like estradiol, | |
| | spermicides), surfactants, | |
| | citric acid, lactic acid | |

As such, the force dissipation and dispersion property of the vulvovaginal sheath 10 will depend upon the combination of material properties that include the force dissipation and dispersion of the material composition in combination with the wall thickness 26, 42 of the vulvovaginal sheath constructed from such material composition. It is anticipated, therefore, that various combinations of material compositions and wall thicknesses can be created to achieve a desired force dissipation ratio. It is further anticipated that different persons suffering from vulvovaginal pain or discomfort may require different force dissipation ratios and/or have a preference for different force dissipation ratios when using the various embodiments of the present invention.

Furthermore, the present invention, in certain embodiments, provides for a vulvovaginal sheath 10 made of a hydrogel material, as described above, wherein the hydrogel material is biodegradable over a period of time when exposed to a combination of temperature and/or friction and/or force and/or pH and/or liquid over a predetermined period of time. The hydrogel is designed in such a way that the degradation rate is controlled so as to not degrade prematurely in the vaginal canal and/or during penetration. A combination of material ingredients from a combination of material ingredient categories can be utilized to provide a hydrogel having material properties that enable it to dissolve or remain intact after a predetermined amount of time in certain conditions. Furthermore, the material ingredients can be utilized such that the hydrogel has a degradation or dissolve rate slow enough to maintain structure and lubricity throughout intercourse or in one embodiment at least 20 min of time after insertion and in another embodiment at least 30 min of time after insertion, and in another embodiment at least 40 minutes or more of time after insertion. Maintaining structural integrity for a predetermined period of time is critical for maintaining its force dispersion and dissipation properties (hence pain-mitigation properties) during use. It is also understood that users may be concerned by any signs of tearing or significant degradation after or during removal. It is also understood that by making the hydrogel removable, the amount of water and ingredients left in the vaginal canal is decreased. As such, when used as a vulvovaginal sheath, such hydrogel would last for a predetermined period to enable the initiation of the act of sexual intercourse, to enable the entire act of sexual intercourse and/or an intermediate predetermined period of time. Such predetermined periods of time may be less than 5 minutes, up to about 10 minutes, up to about 30 minutes and up to and more than 60 minutes depending on the material ingredients. It is anticipated that different people suffering from vulvovaginal pain and/or discomfort will have different preferences and or requirements as to the predetermined period during which the sheath is used. It is important to reiterate that the sheath, in accordance with the embodiments disclosed herein, is lubricious but is differentiated from vaginal suppositories and liquid lubricants. The soft-solid hydrogel is not a liquid (traditional lubricant) nor is it a full solid that is meant to degrade completely in the vaginal canal (suppository). Instead, the hydrogel is a pillowy solid with an outer surface that is capable of being moist and wet whether through its natural state or by introducing an additional layer of hydrogel, and/or water-based lubricant, and/or oil, and/or or emulsion of both water and oil to the surface of the device before or after packaging and/or use and/or combinations thereof. Through this additional lubrication layer, the hydrogel is immediately lubricious and ready to use without additional preparation or lubrication by the user.

There are various embodiments of the sheath 10, including the tubular material structure 20 and/or flange 40 portion material structure in accordance with the present invention. In the embodiment shown in FIGS. 1 and 2, the tubular structure 20 is comprised of a single hydrogel layer, as discussed above, in some embodiments the hydrogel may further incorporate a lubrication layer on the inner and/or outer surfaces of the sheath 10. This lubrication layer may include one or more oils (almond, coconut, shea, jojoba, dimethicone, a mixture of oils, etc.) alone or in combination with a water-based lubrication. In other embodiments, the sheath 10 may comprise a polymer material such as silicone, rubber, latex, thermoplastic, plastic, or polyurethane and may or may not have a layer of lubrication covering an inner surface and/or an outer surface thereof. In yet another alternative embodiment, the sheath 10 may be comprised of a multi-layer hydrogel wherein each hydrogel layer may be comprised of a hydrogel having a different formulation and different material properties. Yet another alternative embodiment is a tubular structure 20 in accordance with the present invention wherein a base layer is comprised of a polymer including, by way of example but not limited to; silicone, polyurethane, rubber, thermoplastic, plastic or latex.

In yet another embodiment, opposing inner wall and outer wall surfaces 22, 24 of the tubular structure 20 each comprise one or two hydrogel layers wherein each hydrogel layer and may comprise different thicknesses, stiffnesses and exhibit various other properties. In another embodiment, the tubular structure 20 and/or flange 40 is a hydrogel layer in combination with one or two hydrogel layers on the inner surface and/or outer surface of the tubular structure of the sheath. It is understood that each of the various embodiments described above may have a layer or layers having any one or more of the various properties such as a layer thickness, layer stiffness, layer force dispersion and dissipation, layer dissolve rate, lubricity, and other material properties suitable for the intended purposes of the present invention.

As shown in FIG. 2, the sheath 10 comprises a fully closed distal portion 34 whereas one or more elongate channels 50 are formed in the inner wall surface 22. The channel or channels 50 extend generally axially along the elongate length of the tubular structure 20. The channels 50 define air pockets to allow air to flow in and out of the proximal portion of inner wall of the sheath 10 along the channel(s) 50. In FIGS. 1 and 2, having an enclosed distal portion 34 with no holes or apertures, the vulvovaginal sheath can prevent sperm and/or semen from passing through the distal portion 34. In the embodiment of a sheath 10 comprising a hydrogel, the hydrogel being a porous material, still has small enough pores to prevent sperm and/or semen from traveling through the hydrogel.

In an alternative embodiment, the tubular structure 20 of the present invention may include a porous section at a distal end 32 thereof, and/or an open distal end portion, for enabling semen to pass through the porous and/or distal end portion of tubular structure 20 during sexual intercourse. The porous end also allows for air to not build up within the sheath 10 during sexual intercourse or penetration. Similarly, another embodiment of the present invention comprises a tubular structure 20 having a single opening at a distal end thereof for enabling semen to pass through the single opening of the tubular member during sexual intercourse. The opening or openings at the distal end can be located anywhere at the distal end, including the curved portion 52. This hole/s in the distal end reduces or eliminates the build up of air being captured within the sheath 10 during sexual intercourse or penetration.

In yet another embodiment, the vulvovaginal sheath 10 has a padded wall thickness 54 at a distal end 32 thereof, the padded wall thickness 54 area having a greater wall thickness (5 mm to 13 mm) than the wall thickness 26 of the tubular structure 20 of the vulvovaginal sheath 10. The padded wall thickness 54 is intended to provide additional force dispersion at the distal end 32 of the vulvovaginal sheath in order to reduce the magnitude of direct collision forces against the cervix and the surrounding anatomical area. The geometry of the pointed end 32 also helps prevent tearing of the sheath 10 during insertion. In an alternative embodiment, the padded wall thickness portion 54 of the distal end 32 may further include an aperture or embody a porous structure while still having an overall padded wall thickness 54 as described above. The dimension of the padded wall thickness portion 54 may be greater than about 1 mm to greater than about 15 mm.

FIGS. 1 and 2 further illustrate a vulvovaginal sheath 10 having an annular bulging portion 60 extending outwardly with respect to the outer wall 24 of the tubular structure 20. The annular portion 60 provides an anchoring function for the sheath 10 within the vaginal canal to minimize movement during penetration. The annular portion 60 is spaced and dimensioned along the sheath 10 to open and relax the muscular tissue when inserted, by applying pressure on the tissue just distal of the pelvic floor muscles.

Also pictured in FIGS. 1 and 2 is a textured surface 62 on the inner wall 22, such textured surface 62 includes one or more recesses 64 such as the ribbed recesses shown in FIGS. 1 and 2. It is understood that such textured surface 62 may be annularly recessed ribs, as shown, or dimples and/or various shapes and combinations of recesses. The textured surface 62 provides friction for the male partner during intercourse and to maintain pleasure.

Having now discussed various embodiments of a vulvovaginal sheath 10 including but not limited to the tubular structure 20 discussed with reference to FIGS. 1 and 2 and the various material layers and construction of the tubular structure 20 and the porous and/or open embodiments, it is understood by one of ordinary skill in the art that any combination and arrangement of the various embodiments and elements and features thereof may be used alone or in combination with any other such embodiments, elements and features in accordance with the scope and spirit of the present invention.

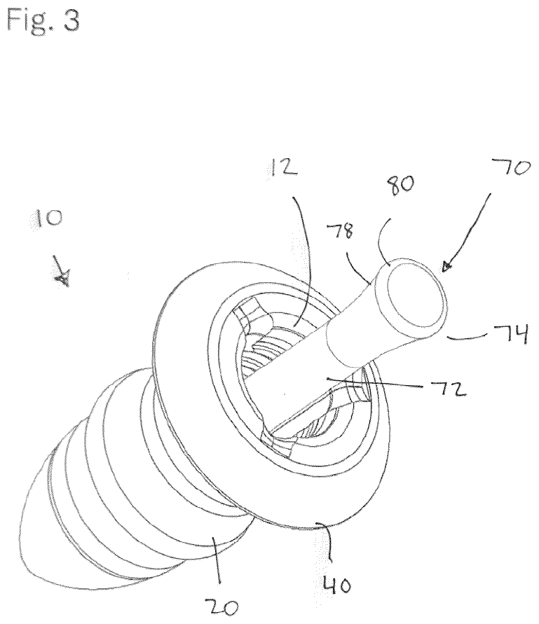

FIGS. 3-5 illustrate various embodiments and elements of an applicator 70 for use in accordance with the present invention. The applicator 70, generally, is used to insert the vulvovaginal sheath 10 into the vaginal canal. The embodiment for an applicator 70 shown provides for a rod or cone shaped structure 72; the structure 72 is rigid in that it is able to maintain its shape when used for its intended purpose, as shall be described below. The structure 72 may be tapered to include a proximal end portion 74 having a proximal diameter and a distal end portion 76 having a distal diameter wherein the proximal diameter is greater than the distal diameter with a tapered portion 78 therebetween. Alternatively, the rod structure 72 may have a fixed diameter along the entire length of the structure. The distal end portion 76 may be rounded to enhance placement. The diameter of the rod structure 72 may be anywhere from about 5 mm to 25 mm in diameter.

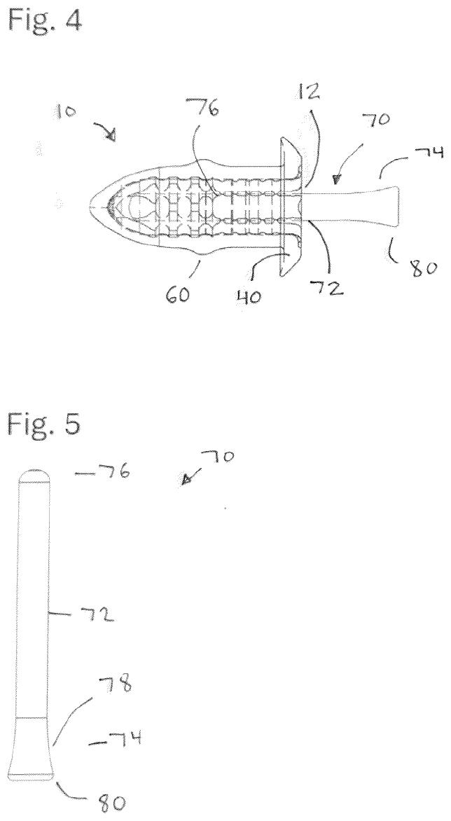

The applicator 70 is used to insert the vulvovaginal sheath 10. This insertion step is performed by loading the vulvovaginal sheath 10 onto the applicator 70, as shown in FIGS. 3 and 4. More specifically, the insertion portion of the applicator 70 is inserted through the opening 12 of the vulvovaginal sheath 10 defined by the inner wall surface 22 of the tubular structure 20. The insertion portion 72 of the applicator 70 may be inserted into the vulvovaginal sheath 10 until the distal portion 76 of the insertion portion is in contact or is proximate to the corresponding inner wall 22 of the distal end portion 32 of the vulvovaginal sheath 10. Once the vulvovaginal sheath 10 is properly loaded onto the applicator 70, the vulvovaginal sheath 10 is inserted into the vaginal canal. The insertion of the vulvovaginal sheath 10 is performed by a user in accordance with a user preference but may generally include grasping of the applicator 70 by the proximal portion 74 and/or handle portion 80 of the applicator 70, depending on the embodiment of applicator 70 being used, the proximal portion 74 being defined as the portion of the applicator 70 not encased in the vulvovaginal sheath 10 after loading. It is understood, however, that a user may prefer to grasp a portion of the exterior of the vulvovaginal sheath 10 or flange 40 during insertion as well. Once a user has grasped the loaded applicator 70, the user may then manipulate the applicator 70 so as to insert the vulvovaginal sheath 10 into the vaginal canal.

In accordance with the various embodiments of the present invention, the user may insert the vulvovaginal sheath 10 into the vaginal canal until reaching a depth at which the flange 40 and/or proximal most portion 28 of the vulvovaginal sheath 10 is in contact with and/or in approximate alignment with the exterior anatomy of the vaginal canal.

Once the vulvovaginal sheath 10 is in place, the applicator is removed 70 from the vulvovaginal sheath 10 while maintaining the positioning of the vulvovaginal sheath 10 within the vaginal canal. Upon removal of the applicator 70, the vulvovaginal sheath 10 is in place to enable sexual intercourse and/or vaginal penetration with the outer flange 40 against the labia. It is important to note that a user having vulvovaginal pain or discomfort is enabled by the method and use of the various embodiments of the present invention to engage in sexual intercourse and/or penetration without the need to first fully complete dilation therapy. It is further important to note that the various embodiments of a vulvovaginal sheath 10 are provided herein to not obstruct or block entry of the vaginal canal or prevent intercourse or penetration. Instead, the various embodiments of the present invention provide for a barrier between the vaginal wall and the penis and or other object entering the opening 12 defined by the vulvovaginal sheath 10, thereby enabling sexual intercourse and/or vaginal penetration while minimizing and/or reducing vulvovaginal pain or discomfort during such activities.

During the activity of sexual intercourse and/or vaginal penetration, and as discussed above with reference to the various embodiments of a vulvovaginal sheath, the material properties of a particular embodiment of the vulvovaginal sheath 10 provide for a dissipation and dispersion of forces against the vaginal walls, cervix, vulva, vestibule, mucosa etc. and thereby reduce vulvovaginal pain or discomfort during the activity. It is understood that different users have different levels of and/or tolerance for vulvovaginal pain or discomfort such that different embodiments of the vulvovaginal sheath will provide different results in the reduction and/or alleviation of vulvovaginal pain or discomfort for different users.

While the above description is illustrative of the various embodiments of the present invention, it is understood that various alternatives and combinations though not explicitly provided herein are further within the scope and spirit of the present invention. For instance, it is anticipated that the sheath may be used as a male masturbator apparatus, or may be used during anal penetration, or may be used as a girth enhancer to a penis or cover on a dilator or sex toy.

Provided below are various examples and embodiments of material compositions and formulations for use in accordance with the various embodiments of the present invention.

### Example 1: Hydrogel Vaginal Sheath Composition and Formulation

TABLE 1
Konjac

| | Concentration | | | |
|---|---|---|---|---|
| Compound | (% w/w) | Use | Must | Optional |
| Carrageenan and | 1-6 | Hydrogel | y | |
| Konjac Gum | | Texture | | |
| Mixture | | | | |
| Glycerin | 0-10 | Hydrogel | | y |
| | | Texture | | |
| Phenoxyethanol and | 0-1 | Preservative | | y |
| Ethylhexylglycerin | | | | |
| Mixture | | | | |
| Sodium Benzoate | 0-.5 | Preservative | | y |
| Potassium Sorbate | 0-.5 | Preservative | | y |
| Potassium Chloride | 0-1 | Crosslinking | | y |
| | | Agent | | |
| Calcium Chloride | 0-1 | Crosslinking | | y |
| | | Agent | | |
| Citric Acid | 0-1 | pH Modifier | | y |
| Water | 79-99 | Solvent | y | |

### Example 2: Hydrogel Vaginal Sheath Composition and Formulation

TABLE 2
Locust Bean

| | Concentration | | | |
|---|---|---|---|---|
| Compound | (% w/w) | Use | Must | Optional |
| Carrageenan and | 1-6 | Hydrogel | y | |
| Locust Bean Gum | | Texture | | |
| Mixture | | | | |
| Glycerin | 0-10 | Hydrogel | | y |
| | | Texture | | |
| Phenoxyethanol and | 0-1 | Preservative | | y |
| Ethylhexylglycerin | | | | |
| Mixture | | | | |
| Sodium Benzoate | 0-.5 | Preservative | | y |
| Potassium Sorbate | 0-.5 | Preservative | | y |
| Potassium Chloride | 0-1 | Crosslinking | | y |
| | | Agent | | |
| Calcium Chloride | 0-1 | Crosslinking | | y |
| | | Agent | | |
| Citric Acid | 0-1 | pH Modifier | | y |
| Water | 79-99 | Solvent | y | |

In some instances, Example 2 is a preferred embodiment.

### Example 3: Non-Hydrogel Vaginal Sheath Composition and Formulation

TABLE 4
Polymer

| | Shore A | | | |
|---|---|---|---|---|
| Compound | Hardness Scale | Use | Must | Optional |
| Silicone, TPE, | 1-30A | Device | y | |
| plastic, | | Structure | | |
| polyurethane, latex, | | | | |
| rubber, soft | | | | |
| polymer | | | | |

The method of making of a vulvovaginal sheath 10 in accordance with the various embodiments of the present invention include the steps of: combining the compounds a predetermined formulation in accordance with the formulations or ranges or variations thereof provided in the examples above; heating, as required of the formulation material in order to insert and shape the material in a mold; inserting the material into the mold and allowing the material to form into a sheath in accordance with the one or more embodiments of the present invention. The mold includes a molding cavity for forming the outer surface of the sheath and a mold insert for forming the inner surface of the sheath. In the embodiments that include multiple material layers and/or lubrication layers, such molding process and/or lubrication process may be performed as an additional or separate step, as is readily understood by one of ordinary skill in the art.

It is understood that the above description is illustrative of the various embodiments of the present invention and not limiting as to the variations and modifications that one of ordinary skill in the art would understand to be within the scope and spirit of the present invention. Having described the preferred aspects and implementations of the present disclosure, modifications and equivalents of the disclosed concepts may readily occur to one skilled in the art. However, it is intended that such modifications and equivalents be included within the scope of the claims which are appended hereto.

What is claimed is:

1. An insertable vaginal sheath for the alleviation of vulvovaginal pain during sexual intercourse, the insertable vaginal sheath comprising:
a) a tubular structure having an inner wall surface and an outer wall surface;
b) the tubular structure further comprising a wall thickness between the inner wall surface and the outer wall surface; and
c) the tubular structure further comprises a hydrogel material comprising at least 80% water.

2. The vaginal sheath according to claim 1, wherein the tubular structure further comprises a proximal end portion wherein a flange extends outwardly from a proximal end portion of tubular structure.

3. The vaginal sheath according to claim 1, wherein the wall thickness of the tubular structure is at least 1 mm.

4. The vaginal sheath according to claim 1, wherein the inner wall surface further comprises a recessed channel extending from a proximal portion of the inner wall surface of the tubular structure to a distal portion of the inner wall surface of the tubular structure.

5. The vaginal sheath according to claim 1, wherein the inner wall surface of the tubular member further comprises a textured surface.

6. The vaginal sheath according to claim 1, wherein the outer wall surface further comprises an annular ring extending outwardly from a periphery of a central portion of the tubular structure.

7. An insertable apparatus for the alleviation of vulvovaginal pain, the insertable apparatus comprising:
a) a tubular structure comprising an inner wall surface and an outer wall surface, wherein:
b) the outer wall surface is dimensioned to be insertable within a vaginal canal and, upon insertion, to frictionally engage a surface of the vaginal canal during sexual intercourse;
c) the inner wall surface is dimensioned to be capable of enabling penetration therein during sexual activity; and
d) the tubular structure further comprising a force diffusion and dispersion property comprising a predetermined combination of a material composition of the tubular structure and the wall thickness of the tubular structure the predetermined combination being capable of alleviating vulvovaginal pain during sexual intercourse.

8. The insertable apparatus according to claim 7, wherein the wall thickness of the tubular structure is at least 1 mm and the material of the tubular structure is a hydrogel.

9. The insertable apparatus according to claim 8, wherein the tubular structure further comprises an annular ring extending outwardly from a periphery of the outer wall surface of the tubular structure.

10. The insertable apparatus according to claim 9, wherein the tubular structure further comprises a recessed channel extending along at least a portion of the inner wall surface.

11. The insertable apparatus according to claim 10, wherein the tubular structure further comprises a textured surface on the inner wall surface thereof.

12. The insertable apparatus according to claim 11, wherein the textured surface is a ribbed pattern.

13. The insertable apparatus according to claim 12 wherein the tubular structure further comprises a lubrication layer on the outer wall surface.

14. The insertable apparatus according to claim 10, wherein the material is a non-hydrogel and the wall thickness is at least 1 mm.

15. The insertable apparatus according to claim 10, wherein the tubular structure further comprises a distal portion defining a receptacle and having contraceptive properties.

16. The insertable apparatus according to claim 10, wherein the material of the tubular structure is a hydrogel that maintains a tubular structure for at least 10 minutes when exposed to one or multiple factors of friction, heat, and vaginal fluids.

17. The insertable apparatus according to claim 7, wherein the hydrogel comprises at least 80% water and a carrageenan and konjac gum mixture.

18. The insertable apparatus according to claim 7, wherein the hydrogel comprises at least 80% water and a carrageenan and locust bean gum mixture.
